(19) United States
(12) Patent Application Publication
(10) Pub. No.: US 2006/0229581 A1
(43) Pub. Date: Oct. 12, 2006

(54) Package for absorbent articles

(76) Inventors: Ibrahim Ulas, Oberursel (DE); Samuel Ombaku Nyambi, Cincinnati, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/102,057

(22) Filed: Apr. 8, 2005

(51) Int. Cl. A61F 13/15

(52) U.S. Cl. 604/385.02

(57) ABSTRACT

The present invention encompasses a package comprising a container comprising at least one sheet of sheet material having an interior surface and an exterior surface. The interior surface defines an interior space. A plurality of articles is disposed within the interior space. The articles typically have a predetermined characteristic. The container has a bottom end and a top end. The top end has a drawing means partially enclosed in a peripheral channel/hem. A signal is disposed on the drawing means. The signal corresponds to the predetermined characteristic of the article.

### FIELD OF THE INVENTION

The present invention relates to a package for containing and dispensing absorbent articles.

### BACKGROUND OF THE INVENTION

There has been a proliferation of sizes, shapes, absorbency, conformations, and brands in the field of disposable absorbent articles. Thus, users of absorbent articles are presented with a difficult task to differentiate between the many types of articles and the variations of article characteristics. Differentiation and selection of articles is also particularly difficult when the artwork, color, and/or shape of the package changes. In addition, differentiation and selection of catamenial articles on store shelves is particularly difficult due to the many choices of article absorbencies such as light absorbency, regular absorbency, and extra absorbency and article configurations such as daytime articles, nighttime articles, winged versions and the like.

One problem for manufacturers of absorbent articles is that a well designed article can be deemed by consumers to have poor performance when the consumer selected the wrong product. For example, a consumer may select a low-absorbency catamenial article for use on a high flow day, which can result in considerable dissatisfaction.

In a retail setting, purchasers are usually unable to visually inspect the contents of the package to determine the size, shape, and bulk of absorbent articles such as baby diapers, sanitary napkins, and adult incontinence garments. Some consumers may resort to opening the package to visually inspect the article. This practice is particularly dissatisfying to retail outlets because open packages often cannot be sold.

Proper selection of consumer articles requires explicit labeling and/or instructions. Despite considerable attention being given to such matters, mistakes continue to be made by consumers. In some instances, the consumer may be inattentive, unable to determine the characteristics of particular articles, or may have a limited amount of time to make a selection of a given article. In others, the consumer may be shopping with young children who distract the consumer at the point of selection. In others, linguistic difficulties may contribute to improper selection and usage.

Typically, instructional matter pertaining to the proper selection and use of absorbent articles comprises printed text, pictures, diagrams, labels, and combinations thereof. The objective of any optimal instructional matter is to be univocal by conveying a message regarding proper selection and usage in such a clear, concise, and exact manner such that essentially any user, regardless of distractions or adverse conditions, is prompted to choose and employ the article correctly.

Color is often used to convey a particular performance characteristic of a given article. For example, at least one manufacturer of catamenial tampons uses color based signals on both the outer package and the wrapper of such tampons to denote absorbent capacity. In such articles, different colors are used to represent different article characteristic levels of absorbency. For example, a green band on the package and wrapper might signal a super absorbency tampon and blue band might signal a regular absorbency tampon.

There is a continuing unaddressed need for devices for helping the consumer select the right absorbent article for the appropriate circumstances. The problem is complicated by the wide variety of absorbent articles available in the marketplace and the limitations imposed on consumers shopping in a retail environment.

Additionally, there is a continuing unaddressed need for devices that the consumer interacts with for helping the consumer select the right absorbent article for the circumstances.

Furthermore, there is a continuing unaddressed need for devices that can be easily manufactured.

Furthermore, there is a continuing unaddressed need for devices upon which can be placed a variety signals to the consumer.

Actually, there is a continuing unaddressed need for an easy and intuitive package for selecting the proper absorbency, which provides a consumer benefit and ensures that the right article will be used.

There is also a continuing unaddressed need for a package for absorbent articles which can be easily and conveniently handled while reclosing it after having removed the first absorbent article therefrom. There is an additional need for such a package which can be stored and carried in more hygienic conditions. Finally there is a further need for such a package which can be manufactured in a simple and more cost effective manner.

### SUMMARY OF THE INVENTION

The present invention encompasses a package comprising a container comprising at least one sheet of sheet material having an interior surface and an exterior surface. The interior surface defines an interior space. A plurality of articles typically having predetermined characteristics is disposed within the interior space. The container has a bottom end and a top end. The top end has a drawing means, preferably a drawing ribbon or draw string, partially enclosed in a peripheral hem. A signal is typically disposed on the drawing means of a predetermined characteristic of the performance of the article. Said package is according to claims 1 or 7 as claimed.

### BRIEF DESCRIPTION OF THE DRAWINGS

FIG. 1 is a partially cut away perspective view of one embodiment of a container of the present invention.

FIG. 2 is a perspective view of a container having a drawing ribbon pulled and the container in a closed state.

FIGS. 3A and 3B are perspective views of a package of another embodiment of a container of the present invention.

FIG. 4 is a perspective view of a container having a draw string pulled and the container in a closed state.

### DETAILED DESCRIPTION OF THE INVENTION

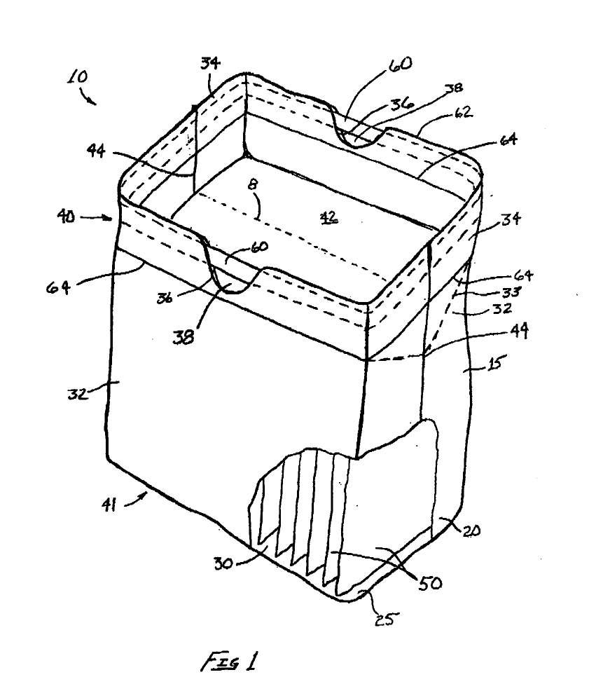

Referring to FIG. 1, one embodiment of the invention shown in partial cut away is a flexible container 10 made from polymer film sheet material 15. Sheet material 15 has an interior surface 25 and an exterior surface 20. The interior surface 25 defines an interior space 30 which is an enclosed interior space when the container is closed, as shown in FIG. 1. The container has a top end 40 and a bottom end 41 and side portions 32. The container may be any shape known in the art. For example, the container may have a polyhedral shape defining or forming a polyhedral enclosure. The articles 50 disposed within the interior space 30 may all be identical to one another or may be different from one another. As shown in FIG. 1, the articles 50 are preferably disposable absorbent articles, such as flat or folded sanitary napkins or pantiliners in a stacked configuration.

The container 10 can be sealed or otherwise closed for shipping and marketing, and typically has an opening means for opening or otherwise accessing the interior space 30 for easy access to the articles 50. As shown in FIG. 1, the opening mechanism or means can be a line of weakness, such as a line of perforations 8. Such line can have any configurations/shapes, e.g. straight or curved. In other alternative embodiments, the opening means for accessing the interior space 30 can be tabs and adhesive openings. The opening mechanism or means for accessing the interior space 30 can be any means known in the art.

The sheet material 15 may be composed of different materials or may be composed of substantially the same type of material. The sheet material 15 may be composed of a laminate. The sheet material 15 can be any flexible material. The sheet material 15 can comprise a blown or cast film of polyethylene, or a blend of low density polyethylene and linear low density polyethylene, or other polymers suitable for flexible films, including metallocene, ethylene vinyl acetate, surlyn, polyethylene terephtalate, biaxially oriented polypropylene, and/or nylon. The sheet material 15 can be cloth. The sheet material 15 can be a nonwoven material. The sheet material 15 can be a woven material.

The absorbent articles 50 of the present invention can be of any type contained within a single container. The absorbent articles 50 preferably are disposable absorbent articles such as diapers, catamenial devices, tampons, sanitary napkins, pantiliners, or adult incontinence products. The articles 50 might also be tissue, wipes and other hygienic absorbent articles.

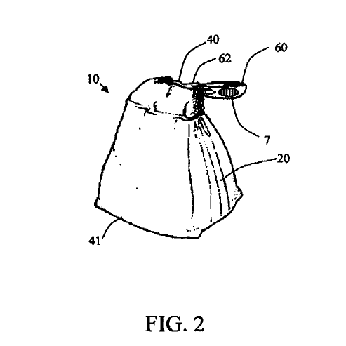

As shown in FIG. 1 top end 40 of container 10 can have a top panel 42 and extensions 34 of sides 32, the extensions 34 extending above top panel 42 when container is oriented as shown in FIG. 1, with the top of the container 10 being at the top of the page. Side extensions 34 comprise a pocket or channel 36 formed by folding over the sheet material 15 to form top edge 62. A seam 64, such as a thermally-sealed line can seal the channel 36. Inside channel 36 can comprises a drawing means such as a drawing ribbon 60. Drawing means can be disposed to move freely inside channel 36 such that the top end 40 of container 10 can be drawn closed as shown in FIG. 2. Drawing means can be constrained to limit lengthwise movement within said channel, typically at at least one location, preferably 2 locations within said channel such as at the seems 44 on opposing side portions 32. Portions of side extensions 34 can be removed to permit user access to drawing means. Said portions can be of any numbers, shape and size.

In use, the user can unseal the package by tearing the line of weakness or line of perforations 8, remove an article from inside the interior 30 of the container 10, and then re-close the bag by pulling on the drawing means such as the drawing ribbon 60 in an easy and convenient manner. Once closed, the drawing means can be used as a convenient carrying handle. Thus in its broadest embodiment the present invention also encompasses a package as claimed in claim 1.

Container 10 can be made by methods known in the art, such as by known “flow wrap” processes in which a web of material is conveyed, cut, folded, seamed, filled and closed. Flow wrap processes can produce generally “cube-shape” or polyhedral-shaped packages by providing a gusset, indicated as 33 in FIG. 1. Gussets 33 permit excess material to be provided for and folded flat. In particular, seams 44 can be made in a flow wrap process for making polymer film bags by a use of a sealing bar that thermally melts and seals container 10 on opposing side portions 32. In such a manufacturing process, drawing means 60 is also thermally sealed at the location of the seam 44 inside channel 36. In a preferred embodiment it is believed to be beneficial to immobilizing the drawing means at these two locations, thereby enabling easier closing of container 10. Instead of sliding lengthwise inside channel 36, the drawing means is constrained, typically at least at two locations, to prevent such lengthwise sliding and to permit closing of container 10 upon being drawn by a pulling force.

In one embodiment herein as shown in FIG. 2, the drawing means is a drawing ribbon 60. The drawing ribbon can be pulled, thereby gathering in the top end edge 62. When the drawing ribbon 60 is pulled to its substantially maximum amount, the top end 40 of the container 10 can be closed such that the articles 50 contained within cannot fall out. The drawing ribbon in a pulled position is an easy and convenient means for the consumer to carry the container 10.

The drawing ribbon 60 can be made of any thin flexible material. The drawing ribbon 60 can comprise a blown or cast film in a blend of low density polyethylene and linear low density polyethylene, metallocenes, ethylene vinyl acetate, surlyn, polyethylene terephtalate, biaxially oriented polypropylene, and/or nylon. The drawing ribbon 60 can be cloth. The drawing ribbon 60 can be a nonwoven material. The drawing ribbon 60 can be a woven material.

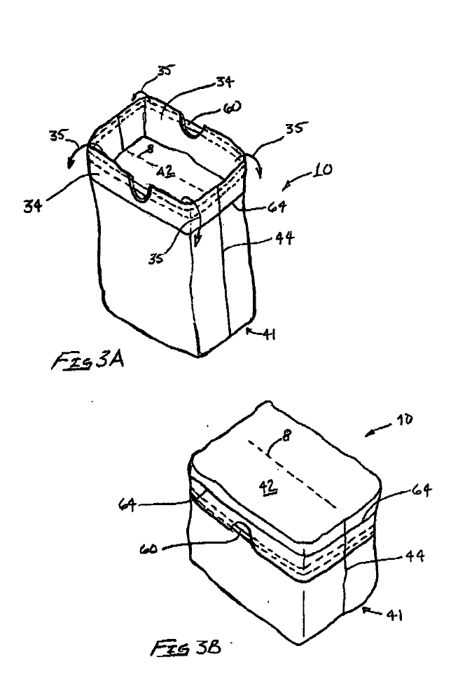

To permit better stacking, storing, packing, or shelving, container 10 can be configured as shown in FIG. 3B. As shown in FIG. 3A, the container as described in FIG. 1 can be modified by folding down side extensions 34 in the direction shown by arrows 35. By folding side extensions 34 all the way down so as to lie substantially flat against sides 32, container 10 can take on a shape having a relatively flat bottom 41 and top 42. Such a configuration enables more stable stacking and shelving, providing a better in-store appearance to consumers. Once opened, for example, once line of perforation 8 is opened, side extensions 34 can be pulled up into the configuration shown in FIG. 3A and drawing ribbon 60 utilized as described above for closing container 10.

In a manufacturing process, the configuration shown in FIG. 3B can be achieved by folding back side extensions 35 prior to side sealing seams 44. Once side extensions 35 are folded back and side seams 44 are made, the process can proceed to container filling and closing.

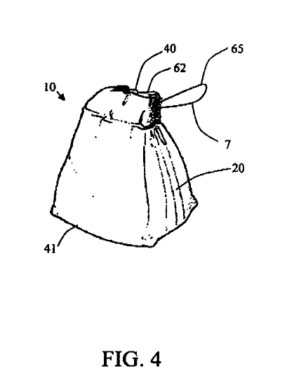

In another embodiment of the invention, the top end 40 has a draw string 65 instead of a drawing ribbon 60. As with the drawing ribbon 60, the drawstring 65 can be pulled, thereby gathering in the top end edge 62, as shown in FIG. 4. When the drawstring 65 is pulled to its substantially maximum amount, the top end 40 of the container 10 can be closed such that the articles 50 contained within cannot fall out. The drawstring 65 in it pulled position can also be an easy way for the consumer to carry the container 10. Draw string 65 can be any of known stings, yarns, fibers, threads, cords, other flexible, string-like elements.

In one embodiment, the present invention provides for signals for use with articles and the packaging for those articles. Signals can provide an easy and intuitive method for selecting the proper package. For example, a signal may be related to the absorbency of an absorbent article within the container 10.

Without being bound by theory, it is believed that by placing the signal to the user on a part of the package that the user physically interacts acts with when opening, closing, and carrying the container, the mental association between the signal and predetermined characteristics of the articles is strengthened, making it more likely that the consumer will select the appropriate article in the future.

In one embodiment, the signal 7 can be color. The term “color” as used herein, relates to the phenomenon of visual perception that enables one to differentiate otherwise identical objects. Colors may be expressed in terms of “hue,” i.e., that attribute of colors that permits them to be classified as red, yellow, green, blue, etc., or as an intermediate color between any pair of colors. The signal 7 can be a color disposed on the drawing means such as the drawing ribbon 60 or drawstring 65 or the material comprising the drawing ribbon 60 or drawstring 65 can itself be colored. The color may be placed on the drawing means (60 or 65) in any shape or pattern such as droplets, circles, parallelepipeds, or the like. The entire drawing means (60 or 65) may be colored or only a portion of the drawing means may be colored.

In another embodiment, the signal 7 can be text. For example, when the container 10 is in the open position, the exposed portion of the drawing means such as drawing ribbon 60 may have text disposed thereon. The text may be disposed on the entire drawing ribbon 60 or disposed on only a portion of the drawing ribbon 60. The same text can be repeated along the entire drawing ribbon 60. Text can identify size, absorbency, use instructions, manufacturer's logo, or any other predetermined characteristic of the articles 50 contained therein. The text can be an inspirational message or any other information that can be communicated via text.

In another embodiment, the signal 7 can be a graphic design. For example, when the container 10 is in the open position, the exposed portion of the drawing means such as drawing ribbon 60 may have a graphic design disposed thereon. The graphic design may be disposed on the entire drawing ribbon 60 or only a portion of the drawing ribbon 60. The same graphic design can be repeated along the entire drawing ribbon 60. The graphic design can identify size, absorbency, use instructions, manufacturer's logo, or any other predetermined characteristic of the articles 50 contained therein. The graphic design can be an inspirational message or any other information that can be communicated via a graphic design.

An article's performance characteristic is the consumer recognition of the execution of a particular characteristic of the article. As used herein, the term “characteristic” refers to any distinguishing trait, quality, or properties of the article. Within the context of this description, an article's performance characteristic can be indicated to the user or consumer by the signal disposed on the drawing means (60, 65). For example, the color of the drawing means of a package of sanitary napkins may be orange to indicate that the sanitary napkins enclosed therein have high absorbency. The orange drawing means allows the consumer to easily identify the absorbency of a given sanitary napkin. The signal may be conveyed to the consumer in a retail environment or at home when the package may already be opened. Text and graphic designs can also be the medium through which the signal to the consumer is conveyed.

### EXAMPLE 1

Example 1 is a package wherein the articles 50 contained therein can be tampons. The sheet material 15 can be low density polyethylene. The container 10 can have a drawing ribbon 60 that is colored purple. The channel 36 can have two openings 38 on opposite sides of the container 10 so that when the drawing ribbon 60 is pulled, the container 10 can be closed, thereby retaining the tampons. The signal 7 can be the color purple and can be indicative of regular absorbency, wherein regular absorbency is deemed to be between 6 and 9 grams.

### EXAMPLE 2

Example 2 is a package wherein the articles 50 contained therein can be multiple diapers. The sheet material 15 can be linear low density polyethylene. The container 10 can have a drawstring 65 that is colored green. The channel 36 can have a single opening 38 through which the drawstring 65 can be drawn. When the drawstring 65 is drawn, the container 10 closes, thereby retaining the diapers. The signal 7 can be the color green and can be indicative of a diaper designed to have extra absorbency so that the diaper can be worn by an infant overnight without leakage occurring.

### EXAMPLE 3

Example 3 is a package wherein the articles 50 contained therein can be sanitary napkins. The sheet material 15 can be biaxially oriented polypropylene. The container 10 can have a drawing ribbon 60 on which the text signal “SuperPlus” can be disposed. The channel 36 can have a single opening 38 through which the drawing ribbon 60 can be drawn. When the drawing ribbon 60 is drawn, the container 10 closes, thereby retaining the sanitary napkins. The signal 7 can be the text “SuperPlus” and can be indicative of a sanitary napkin having a high level of absorbency.

### EXAMPLE 4

Example 4 is a package wherein the articles 50 contained therein can be interlabial catamenial devices. The sheet material 15 can be biaxially oriented polypropylene. The container 10 can have a drawing ribbon 60 on which the signal 7 can be a graphic design resembling a red rose. The signal 7 resembling a red rose can appear repetitively on the drawing ribbon 70. The channel 36 can have a single opening 38 through which the drawing ribbon 60 can be drawn. When the drawing ribbon 60 is drawn, the container 10 closes, thereby retaining the interlabial catamenial devices. The signal 7 can be the graphic design resembling a red rose and can be indicative of high absorbency interlabial catamenial devices.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A package comprising:
a container comprising at least one sheet of sheet material having an interior surface and an exterior surface, said interior surface defining an interior space;
a plurality of absorbent articles disposed within said interior space;
said container having a bottom end and a top end, and,
at or near said top end said package has a channel, said channel at least partially enclosing a drawing means, said drawing means being longitudinally constrained to limit lengthwise movement within said channel.

2. The package of claim 1 wherein said drawing means is a drawing ribbon or draw string.

3. The package of claim 2, further characterized by a signal disposed on said drawing ribbon or a draw string, said signal corresponding to a predetermined characteristic of said absorbent articles.

4. The package of claim 3 wherein said signal comprises a graphic design.

5. The package of claim 3 wherein said signal comprises color.

6. The package of claim 3 wherein said signal comprises text.

7. A package comprising:
a container comprising at least one sheet of sheet flexible polymeric film material having an interior surface and an exterior surface, said interior surface defining an enclosed interior space;
a plurality of absorbent articles disposed within said interior space, said articles having a predetermined characteristic;
said container having a bottom end and a top end;
said top end having a drawing means partially enclosed in a channel; and,
a signal disposed on said drawing means, said signal corresponding to said predetermined characteristic of said articles.

8. The package of claim 7 wherein said drawing means is a drawing ribbon or draw string.

9. The package of claim 7 wherein said signal comprises color, text and/or a graphic design.

10. The package of claim 7 wherein said absorbent articles are disposable absorbent articles selected from the group consisting of tampons, diapers, sanitary napkins, pantiliners, adult incontinent products, interlabial devices, tissue, wipes and combinations thereof.

11. The package of claim 1 wherein said absorbent articles are disposable absorbent articles selected from the group consisting of tampons, diapers, sanitary napkins, pantiliners, adult incontinent products, interlabial devices, tissue, wipes and combinations thereof.
